(19) United States
(12) Patent Application Publication
(10) Pub. No.: US 2021/0307731 A1
(43) Pub. Date: Oct. 7, 2021

(54) MEDICAL SWAB

(71) Applicant: Northwestern University, Evanston, IL (US)

(72) Inventors: Matthew Grayson, Evanston, IL (US); Jun Peng, Evanston, IL (US); Jiaxing Huang, Wilmette, IL (US); Guillermo Ameer, Iowa City, IA (US); Nancy Rivera Bolanos, Evanston, IL (US)

(21) Appl. No.: 17/221,200

(22) Filed: Apr. 2, 2021

Related U.S. Application Data
(60) Provisional application No. 63/039,035, filed on Jun. 15, 2020.
(60) Provisional application No. 63/004,127, filed on Apr. 2, 2020.

(51) Int. Cl. A61B 10/00

(52) U.S. Cl. CPC A61B 10/0051

(57) ABSTRACT

A medical swab includes a hollow tube shaft that has a distal end and a proximal end. A swab tip is mounted to the hollow tube shaft such that a first portion of the swab tip is positioned within the distal end of the hollow tube shaft and a second portion of the swab tip extends out from the distal end of the hollow tube shaft.

### CROSS-REFERENCE TO RELATED APPLICATIONS

The present application claims the priority benefit of U.S. Provisional Patent App. No. 63/004,127 filed Apr. 2, 2020 and of U.S. Provisional Patent App. No. 63/039,035 filed on Jun. 15, 2020, the entire disclosures of which are incorporated by reference herein.

### BACKGROUND

A medical swab refers to a type of swab that is used to collect a clinical sample of nasal secretions from the anterior nares or front nasal passage of a patient, or saliva from the dorsum or body of the tongue of a patient. The clinical sample can be analyzed (typically in a laboratory) to detect the presence of organisms or other markers that indicate disease. This diagnostic method can be used to detect a variety of illnesses/diseases, including whooping cough, diptheria, influenza, and various types of coronaviruses such as SARS, MERS, COVID-19, etc.

### SUMMARY

An illustrative medical swab includes a hollow tube shaft that has a distal end and a proximal end. A swab tip is mounted to the hollow tube shaft such that a first portion of the swab tip is positioned within the distal end of the hollow tube shaft and a second portion of the swab tip extends out from the distal end of the hollow tube shaft.

An illustrative method of making a medical swab includes shaping at least a portion of a material to form a first portion of a swab tip. The method also includes forming a plurality of strips in an end of the material to form a second portion of the swab tip. The method further includes placing the first portion of the swab tip into a distal end of a hollow tube shaft to for the medical swab.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A depicts a medical swab in accordance with an illustrative embodiment.

FIG. 1B is a partial cross-sectional view of the hollow tube depicting angled tabs mounted to an interior wall and used to help secure the swab tip within the hollow tube in accordance with an illustrative embodiment.

FIG. 1C is a partial cross-sectional view of a distal end of the medical swab in accordance with an illustrative embodiment.

FIG. 2 depicts a package for use in a steam sterilization procedure in accordance with an illustrative embodiment.

FIG. 3 depicts tools and materials that can be used to make a medical swab in accordance with an illustrative embodiment.

FIG. 4 is a flow diagram depicting operations used to assemble a medical swab in accordance with an illustrative embodiment.

FIG. 5A depicts dimensions and principle thread direction of a gauze piece in accordance with an illustrative embodiment.

FIG. 5B depicts the material of FIG. 5A with one end parsed into eight strips in accordance with an illustrative embodiment.

FIG. 5C depicts swab tip material that has been shaped using tweezers in accordance with an illustrative embodiment.

FIG. 5D shows insertion of the swab tip material into the hollow tube in accordance with an illustrative embodiment.

FIG. 5E depicts the completed medical swab in accordance with an illustrative embodiment.

FIG. 6A shows a cut piece of swab tip material in accordance with an illustrative embodiment.

FIG. 6B depicts the swab tip material as folded in accordance with an illustrative embodiment.

FIG. 6C depicts the swab tip material inserted into the hollow tube shaft in accordance with an illustrative embodiment.

### DETAILED DESCRIPTION

During times of medical emergency and pandemic, there is often a shortage of testing materials for viral infections, such as coronavirus (COVID-19), or bacterial infections, such as tuberculosis (TB). Recently, there has been a shortage in the medical swabs used for collecting samples for COVID-19 diagnostics. Additionally, TB infections remain rampant in certain developing economies throughout the world, wherein the need for testing is always high. As a result, during times of mass infection, only a very limited number of tests is conducted per day, and the tests are restricted only to people exhibiting a restricted qualification of heavy symptoms. As an example, on Mar. 24, 2020 during the COVID-19 pandemic, the entire state of Illinois was only able to conduct 1600 tests. Other states and nations have also reported dire shortages in testing supplies that indicate a vanishing window of opportunity to collect data regarding the spread of the virus.

Internationally, in communities such as India, testing on a massive scale can be used to monitor and contain the COVID-19 pandemic and any viral pandemics that may arise in the future, as well as monitor breakouts of bacterial infections like TB. However, if sufficient testing cannot be performed, infected persons (including asymptomatic carriers) go uncounted, leading to inadvertent and unchecked spread of the virus. The only way a pandemic can be systematically stemmed is with a greater knowledge of the scope of the problem, which requires a higher volume of testing encompassing all persons possibly infected, including those who have merely come in contact with infected patients but who are not yet exhibiting symptoms.

Traditional swabs for viral sample collection require that the swab absorber be made of a synthetic polymer, typically rayon, Dacron, or nylon. Other designs have all had a pear-shaped swab, such as a q-tip, where the shaft of the swab is typically made of a thin solid polymer. Other designs include a ‘flocked swab’ that has a pear-shaped solid tip that is surrounded by a very thin layer of synthetic polymer to increase the surface area-to-volume ratio of the absorbent layer. Such traditional swabs are expensive and time-consuming to produce and get into the market, as evidenced by the shortages that have arisen during the COVID-19 pandemic.

Described herein are medical swabs that are made with industrially available medical-grade materials (i.e., materials similar to those used in traditional medical swabs) that are inexpensive and that can be mass produced in a very short period of time. A massive supply of such swabs can be used to help alleviate this critical supply bottleneck from the testing workflow. In addition to being inexpensive and capable of high-speed volume production, the proposed medical swabs can also be scalable in size such that they are usable with any patient.

More specifically, the present disclosure provides a new design of medical swab for collecting and releasing a biological sample into saline liquid for testing. In an illustrative embodiment, the new design includes a tube-shaped applicator and medical gauze as a sponge material. The present disclosure also provides a method of forming the swab by pulling a medical gauze through a tube-shaped applicator using tweezers or another mechanical gripper to a specified length. The present disclosure further provides a method of collecting a biological sample only on the front part of the gauze. A non-absorbing area of the swab tip is designed in one embodiment by hot-pressing a melt zone onto a portion of the gauze that is to be contained within the tube. In another illustrative embodiment, a break point can be placed along an appropriate length in the tube applicator so that after sample collection, swabs can be snapped off to the appropriate length inside a standard test tube for sample transport to the testing site. In another embodiment, the swab tip can be released by pushing a plunger down the center of the tube that is holding the a portion of the swab tip such that it is released into a collection tube. In one embodiment, the proposed swabs can also be autoclaved (sterilized) via steam sterilization following standard sterilization protocols. Alternatively, a different type of sterilization procedure may be used.

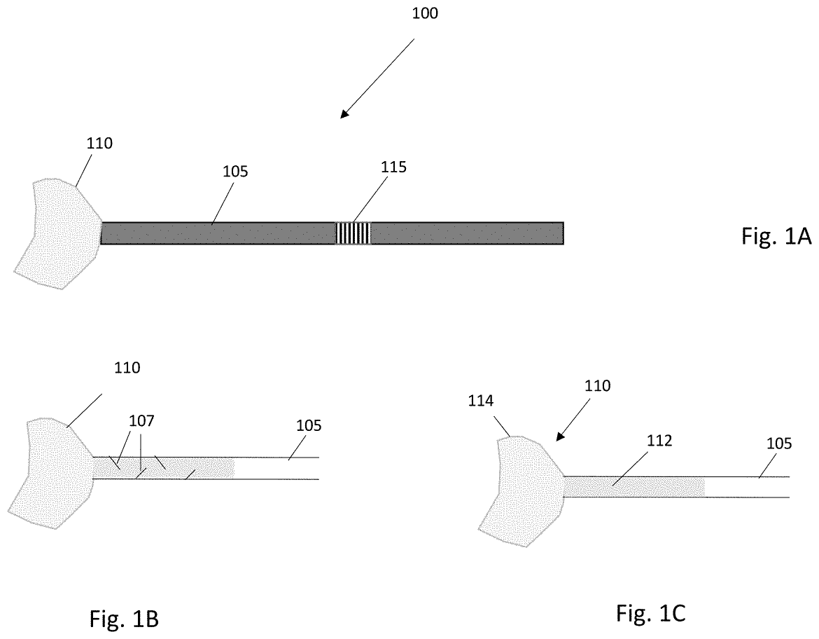

FIG. 1A depicts a medical swab 100 in accordance with an illustrative embodiment. The medical swab 100 includes a hollow tube (or hollow tube shaft) 105, with a swab tip 110 mounted to an end thereof. As used herein, a distal end of the hollow tube 105 refers to the end of the hollow tube 105 to which the swab tip 110 is mounted. A proximal end of the hollow tube 105 refers to the end of the hollow tube 105 that is opposite of where the swab tip 110 is mounted. In one embodiment, the hollow tube 105 can be a polypropylene straw and the swab tip 110 can be made of cotton or synthetic gauze. Alternatively, a different type of material may be used for the hollow tube 105, such as plastic, rubber, polyester, etc. Similarly, in some embodiments a material other than gauze can be used for the swab tip 110 such as other natural fibers, linen, polyester, other synthetic fibers, etc.

In an illustrative embodiment, material that forms the swab tip 110 is drawn through the hollow center of the tube (i.e., starting at the distal end of the hollow tube 105 and moving toward the proximal end of the hollow tube 105) until approximately 2 cm of material extends out of the hollow tube 105 to form the swab tip 110. Alternatively, a different length of swab tip 110 can be used such as 0.5 cm, 1 cm, 3 cm, etc. In other words, the material used to form the swab tip 110 can be inserted into the distal end of the tube 105 and pulled into the tube until approximately 2 cm of the material extends from the distal end of the hollow tube 105 to form the swab tip. The portion of the material (e.g., cotton gauze) within the hollow tube 105 can be secured to the interior wall of the tube using an adhesive such as a glue, caulk, or silicon, by friction fit, by a fastener, by a clip, etc. In one embodiment, a friction fit is used so that the swab tip can be pushed out of the hollow tube 105 with a plunger.

The hollow tube 105 also includes a crimp 115 that provides an easy breaking point such that a user can quickly and easily break off the portion of the medical swab that includes the swab tip 110 and a sample collected thereon. This allows the swab tip 110 to be placed into a standard test tube and capped for sample delivery to the testing site. In one embodiment, the crimping to form the break-point can be achieved by way of a heat press at a temperature just above the melting temperature of the tube shaft, which can be around 170-200 Celsius (C). In alternative embodiments, a different crimping method may be used such as removing one or more portions of the hollow tube (e.g., a circular arrangement of holes about the circumference of the hollow tube), making one or more portions of the hollow tube wall thinner, etc. In other alternative embodiments, a crimp may not be used. For example, in one embodiment, a plunger can be inserted at the proximal end of the hollow tube 105 and pushed toward the distal end until the swab tip 110 is pushed out of the hollow tube 105 and into a test tube or other receptacle. In this way, the risk of contamination can be reduced if the plunger is kept sterilized. In one embodiment, the plunger can be a rod made of rubber, silicon, metal, or plastic, and can be sized to slidably fit within the hollow tube 105. The plunger can be longer than the hollow tube 105 such that the swab tip 110 can be completely ejected out from the distal end of the hollow tube 105.

In one alternative embodiment, the hollow tube 105 can include one more tabs on its internal wall that are angled toward the proximal end of the hollow tube 105. These angled tabs can help to secure the swab tip 110 and prevent it from being easily pulled out of the distal end of the hollow tube 105. FIG. 1B is a partial cross-sectional view of the hollow tube 105 depicting angled tabs 107 mounted to an interior wall and used to help secure the swab tip 110 within the hollow tube 105 in accordance with an illustrative embodiment. Although four angled tabs 107 are shown, additional or fewer tabs may be used in alternative embodiments. Alternatively, the angled tabs 107 may not be used.

In another embodiment, to minimize absorption of an obtained sample into the unused portion of the gauze that resides within the hollow tube 105, the material (e.g., gauze) internal to the hollow tube 105 can be heat-pressed such that the fibers of the material are fused together before insertion into the shaft. The fused fibers resist absorption of the obtained sample such that the majority of the sample remains on the portion of the swab tip 110 that extends out from the distal end of the hollow tube 105. Instead of or in addition to heat-pressing the fibers of the material, in one embodiment, a hydrophobic coating can be applied to the portion of the swab tip 110 that is retained within the hollow tube 105. The hydrophobic coating can be manganese oxide polystyrene, zinc oxide polystyrene, precipitated calcium carbonate, silica nano-coating, etc.

FIG. 1C is a partial cross-sectional view of a distal end of the medical swab in accordance with an illustrative embodiment. As shown, the swab tip 110 includes a first portion 112 that is secured within the hollow tube 105 and a second portion 114 that extends from the distal end of the hollow tube 105. As discussed, the first portion 112 can be heat-pressed and/or have a coating applied such that the first portion 112 resists absorption of a sample that is received on the second portion 114. The first portion 112 can be secured within the hollow tube 105 using an adhesive (that may also act as a hydrophobic coating), by a friction fit, by one or more angled tabs within the hollow tube 105, etc.

In practice, upon insertion into the anterior nares or front nasal cavity of a patient, or upon applying to the tongue of a patient, the swab can be turned to effectively collect a sample onto the swab tip area that extends from the distal end of the hollow tube 105 (i.e., the second portion 114 of the swab tip 110). Similar to a flocked swab, the present design has a very high surface area-to-volume ratio, meaning that the amount of sample collected is proportional to the area of the exposed swab tip 110, and the amount of sample transfer directly to the saline solvent for virus testing is inversely proportional to the volume of the swab tip 110. Swab designs that have a high surface area-to-volume ratio will transfer all of the sample into the saline solution for testing, whereas swabs with a low surface area-to-volume ratio will absorb the sample deeper into the volume of the swab, thereby reducing the testable concentration of virus that reaches the solvent.

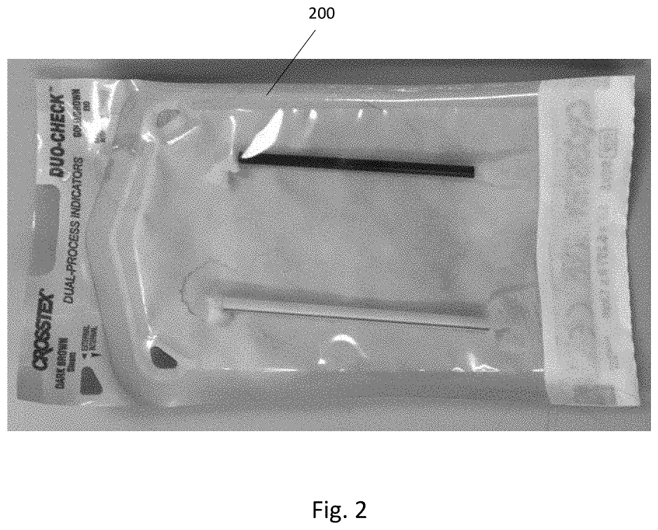

After assembly and prior to use, the medical swabs can be sterilized to help keep the patient safe and also to help prevent false positive (or negative) test results. FIG. 2 depicts a package 200 for use in a steam sterilization procedure in accordance with an illustrative embodiment. The embodiment of FIG. 2 depicts two different swabs within the package 200. In alternative embodiments, fewer or additional swabs may be sterilized using the package 200, such as one swab, three swabs, etc. In the sterilization procedure, swabs are placed into the package 200, and the package 200 is then autoclaved in a steam bath inside of the package 200 for storage and eventual delivery to the test collection site. In one embodiment, the autoclaving can occur in a 120° Celsius (C) steam bath for 50 minutes. Alternatively, a different temperature (e.g., 118° C., 122° C., etc.) and/or amount of time (e.g., 48 minutes, 55 minutes, 60 minutes, etc.) may be used. Additionally, in alternative embodiments a different sterilization procedure may be used.

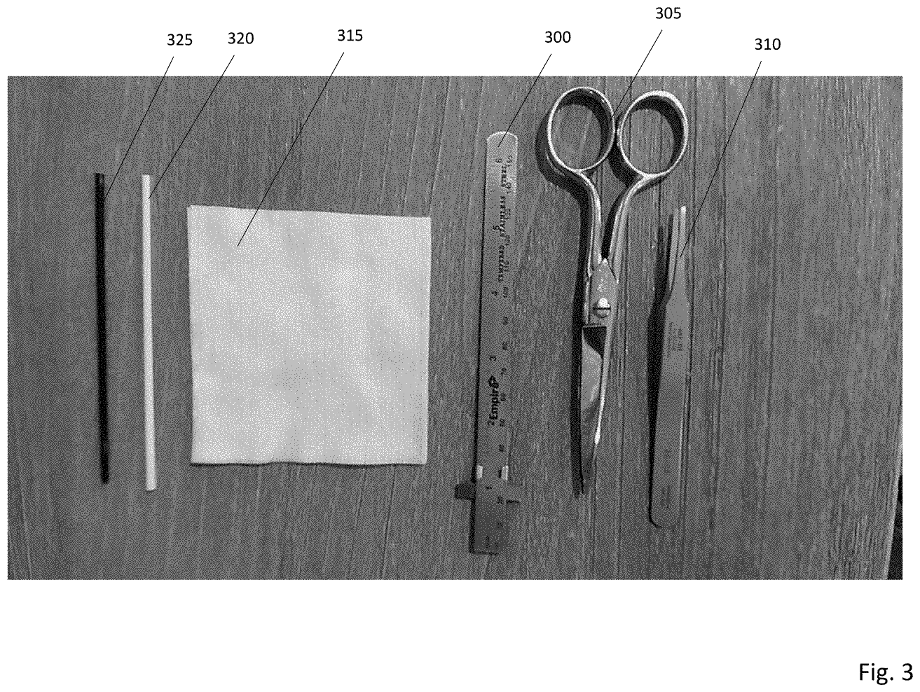

In an illustrative embodiment, the proposed medical swab can be made using simple tools and readily available materials. FIG. 3 depicts tools and materials that can be used to make a medical swab in accordance with an illustrative embodiment. The tools include a ruler 300, a pair of scissors 305, and a pair of tweezers 310 and are used to assemble the materials into a medical swab. The materials include a cloth 315, a straw 320, and a rod 325. In an illustrative embodiment, the cloth 315 can be a 3-ply non-woven synthetic gauze (e.g., Medline) that is 2 inches×2 inches or 4 inches×4 inches. The straw 320 can be 12.5 centimeters (cm) in length and have a 3.5 millimeter (mm) diameter, and the rod 325 can be 12.5 cm in length and have a 3 mm diameter. As such, the rod 325 can fit within the straw 320 and act as a plunger as described herein. In alternative embodiments, the materials may have different dimensions. In other alternative embodiments, fewer, additional, and/or different tools and materials may be used. As just one example, instead of the scissors 305, a knife or razor may be used.

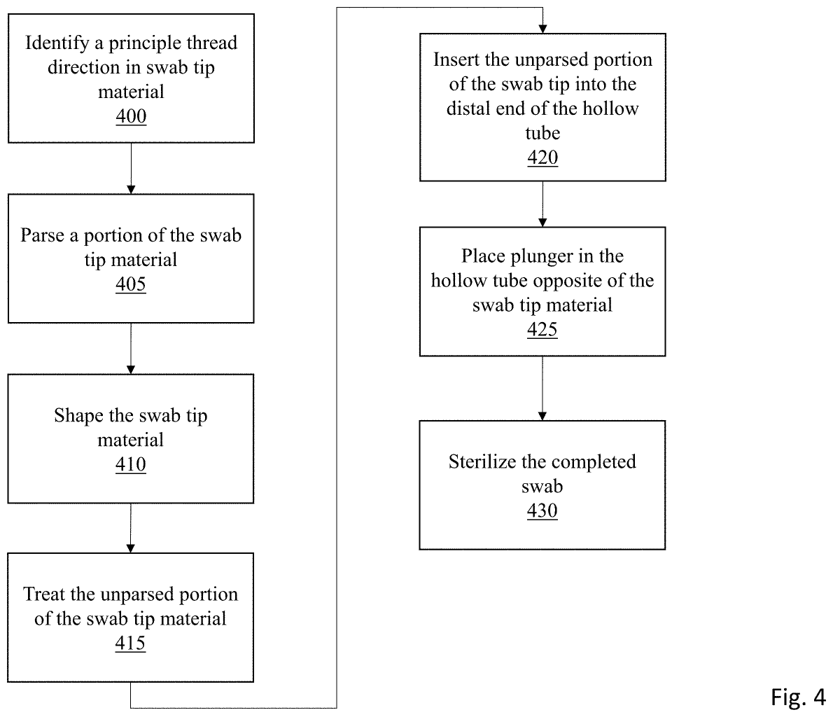

FIG. 4 is a flow diagram depicting operations used to assemble a medical swab in accordance with an illustrative embodiment. In alternative embodiments, fewer, additional, and/or different operations may be performed. Additionally, the use of a flow diagram is not meant to be limiting with respect to the order of operations performed. In an operation 400, the assembler identifies a principle thread direction in material to be used for the swab tip. As discussed in more detail below, the principle thread direction helps the assembler to identify the direction in which the material is strongest and least likely to tear. In alternative embodiments in which the material being used is different, the material may not have threads or a readily identifiable thread direction.

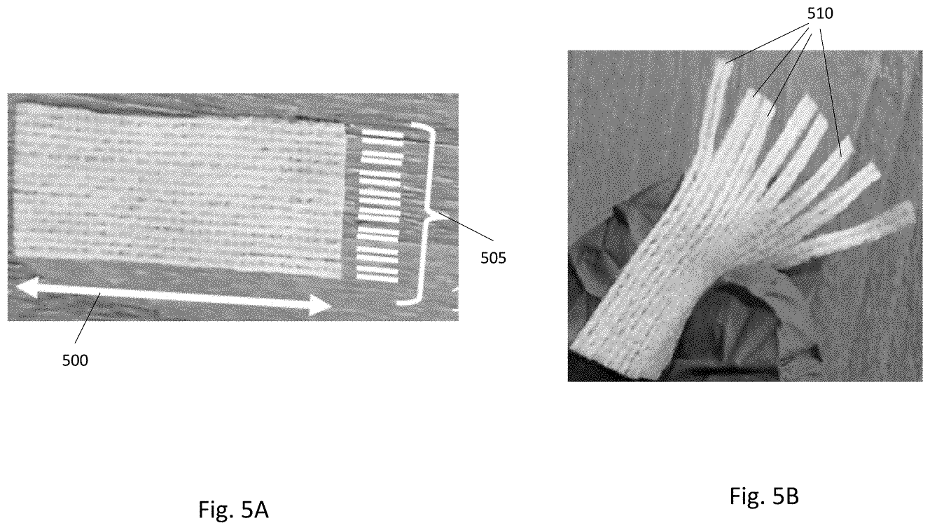

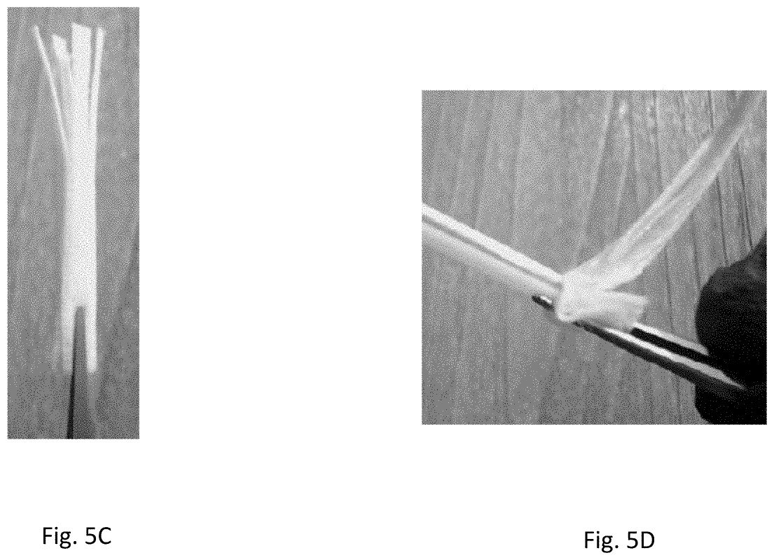

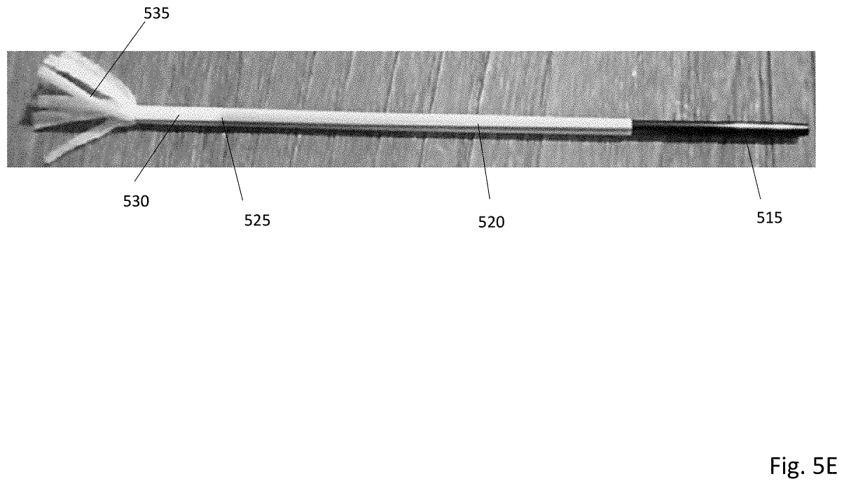

FIG. 5A depicts principle thread direction in a piece of swab tip material in accordance with an illustrative embodiment. The material in FIG. 5 is a non-woven synthetic gauze that has dimensions of 2.4 cm by 5 cm. Alternatively, a different type and/or size of material may be used. An arrow 500 is used to denote the principle direction of the threads used to form the material. As shown by the lines 505, there are 16 threads (i.e., 16 thread count) present in the material shown. In the material of FIG. 5, the long side is thus parallel to the principle thread direction. The assembler can identify the thread direction (500) via a visual inspection of the material. It is important to identify the thread direction because some materials are stronger in one direction than the other. For example, non-woven synthetic gauze is much stronger parallel to thread direction, and weaker perpendicular thereto.

Referring again to FIG. 4, the assembler parses a portion of the swab tip material in an operation 405. In one embodiment, the assembler parses the swab tip material by making one or more cuts to form strips in an end portion of the material. In an illustrative embodiment, the cuts are made parallel to the identified principle thread direction such that each strip of material has maximum strength and is less likely to tear off from the uncut portion of the material. For example, the assembler can parse one end of the material of FIG. 5A by making 7 cuts to create 8 strips that are 2 cm deep. As such, each strip contains 2 threads and is ˜0.3 mm wide. FIG. 5B depicts the material of FIG. 5A with one end parsed into 8 strips 510 in accordance with an illustrative embodiment. In alternative embodiments, a different number of strips, a different length of the strips (i.e., depth of the cut), and/or a different width of the strips may be used.

In an operation 410, the assembler shapes the swab tip material. In an illustrative embodiment, the shaping is performed primarily on the uncut (or unparsed) portion of the swab tip material such that the uncut portion of swab tip material is able to fit into the hollow tube. In one embodiment, the material can be shaped by folding the material in half along its length, and then in quarters, and so on until the desired size and shape are achieved. FIG. 5C depicts swab tip material that has been shaped using tweezers in accordance with an illustrative embodiment. As shown, the shaped material is substantially cylindrical so that it fits within the hollow tube (or straw). As also shown in FIG. 5C, the bottom ˜1 cm of the swab tip material is held by the tweezers once the desired shape is achieved. As discussed below, holding the swab tip material in this fashion can assist with insertion into the hollow tube.

In an operation 415, the assembler treats the unparsed end of the swab tip to reduce its absorption. In one embodiment, treating the unparsed portion can involve heat pressing (i.e., melting) the unparsed and shaped portion of the swab tip material so that the fused/melted material is less absorptive. Treating the material can alternatively involve applying a hydrophobic coating to the unparsed portion of the material so reduce absorption. In an embodiment in which the swab tip material is not intended to be removed from the hollow tube, an adhesive (e.g., glue) can be applied to unparsed portion of the material to both treat the material, making it less absorptive, and also to help the material adhere to the internal wall of the hollow tube. In an alternative embodiment, a treatment may not be applied to the unparsed portion of the swab tip material.

In an operation 420, the assembler inserts the unparsed portion of the swab tip material into the distal end of the hollow tube. In an embodiment in which tweezers are used, the tweezers can be used to hold the shaped swab tip material as shown in FIG. 5C. The material is then folded at the tweezer tip and pushed into the distal end of the wider diameter hollow tube. In one embodiment, the material is positioned within the hollow tube such that the parsed material extends ˜2 cm from the end of the hollow tube to form the swab tip. Alternatively, a different length can extend from the distal end of the hollow tube, such as 1.5 cm, 2.5 cm, 3 cm, etc. FIG. 5D shows insertion of the swab tip material into the hollow tube in accordance with an illustrative embodiment.

In an operation 425, the assembler places the plunger rod into the hollow tube opposite of the swab tip material. Specifically, the plunger rod can be placed into the proximal end of the hollow tube and inserted until the plunger rod is close to (or alternatively in contact with) the unparsed portion of the swab tip material that occupies the distal end of the hollow tube. The plunger rod is as long as the hollow tube or longer so that it can be used to remove the swab tip once a sample has been collected. FIG. 5E depicts the completed medical swab in accordance with an illustrative embodiment. As shown, the plunger rod 515 is inserted into the proximal end of the hollow tube 520 and sticks out past the proximal end of the hollow tube. As also shown, a distal end 525 of the plunger rod 515 stops just short of the unparsed portion 530 of the swab tip, which is inside of the hollow tube 520. The parsed end 535 of the swab tip extends from the distal end of the hollow tube 520.

Referring back to FIG. 4, in an operation 430, the completed swab is sterilized. The medical swab can be sterilized by placing one or more swabs in a package and autoclaving the package. Alternatively, any other sterilization technique(s) may be used. It is noted that during sample collection, the entire stem of the medical swab can be freely handled with bare hands, without any risk of contaminating the sample due to the swab release feature that is enabled by the plunger rod. Specifically, after collecting sample with swab, the swab tip can be released into the collection vessel by pushing the inner plunger rod. This eliminates the need for gloves when performing sample collection, making a self-administered, at-home sampling kit much more feasible.

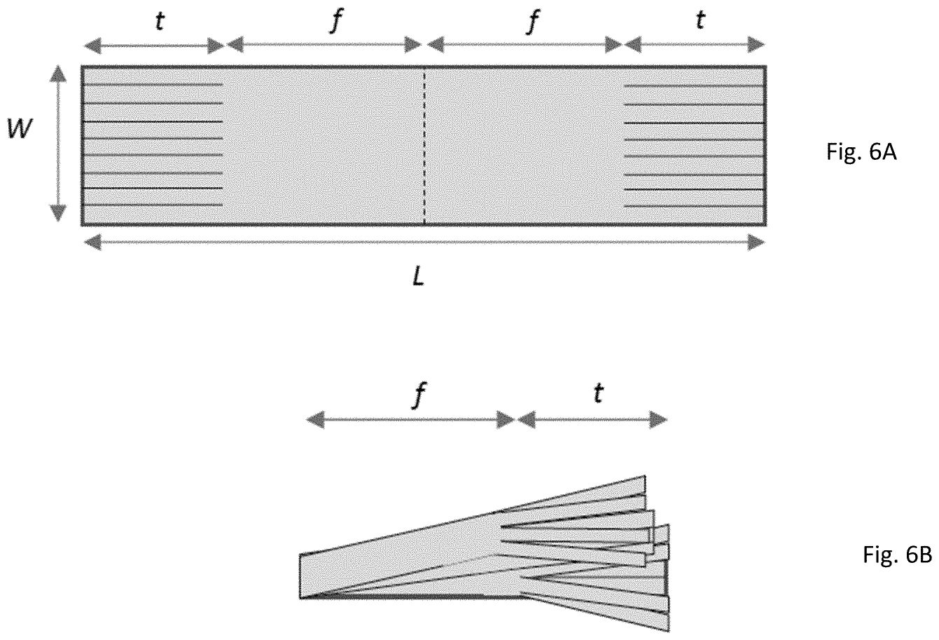

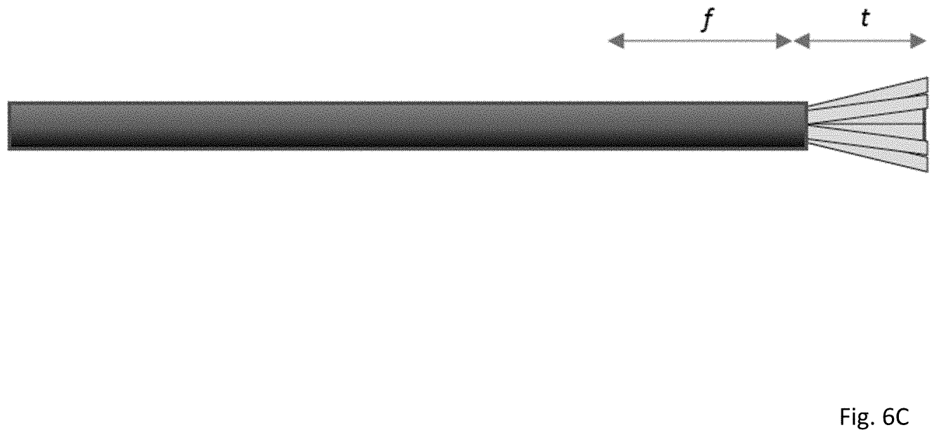

FIGS. 6A-6C depict example generic dimensions for a medical swab. FIG. 6A shows a cut piece of swab tip material in accordance with an illustrative embodiment. FIG. 6B depicts the swab tip material as folded in accordance with an illustrative embodiment. FIG. 6C depicts the swab tip material inserted into the hollow tube shaft in accordance with an illustrative embodiment. The total length of the swab tip material is defined as L, the length of the tassels (or strips) as t and the length of the folded segment within the hollow tube as f. In this design, the swab length is equal to twice the sum of the length of the strips and the folded lengths, L=2f+2t. The symmetry of this design increases viral load collection yield up to another factor of ×2, given the doubling of the number of strips. It is noted that the proposed design above maintains appropriate tension for the swab. All of the data of tensile force F versus folded insertion length f has been found to be valid. In the depicted embodiment, the total length of the gauze is L=2f+2t, with a strip length t=2 cm. The width of the gauze piece is W=2.4 cm in all cases. Various embodiments can be L=7 cm total gauze length, f=1.5 cm fold, L=8 cm total gauze length, f=2.0 cm fold, L=9 cm total gauze length, f=2.5 cm fold, etc. Alternatively, different widths, gauze lengths, and/or strip lengths may be used.

It is expected that the proposed low-cost and rapidly available alternate swab test could significantly alter the pandemic management. By allowing testing not only for symptomatic, but also for mildly symptomatic and even asymptomatic patients who have been in contact with an infected patient, high-volume testing will allow reliable statistics to be gathered as well as prescriptive measures for quarantine to be administered. Urgent deployment will mean the difference between a pandemic that is survivable, versus one that overwhelms the healthcare system with collateral damage on all aspects of healthcare which can no longer function properly under the excessive number of cases.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A medical swab comprising:
a hollow tube shaft that has a distal end and a proximal end; and
a swab tip mounted to the hollow tube shaft such that a first portion of the swab tip is positioned within the distal end of the hollow tube shaft and a second portion of the swab tip extends out from the distal end of the hollow tube shaft.

2. The medical swab of claim 1, wherein the second portion of the swab tip that extends out from the distal end of the hollow tube shaft includes a plurality of strips of material.

3. The medical swab of claim 1, wherein the first portion of the swab tip is at least partially melted to reduce absorption by the first portion.

4. The medical swab of claim 1, further comprising a treatment applied to the first portion of the swab tip to reduce absorption by the first portion.

5. The medical swab of claim 4, wherein the treatment comprises a hydrophobic coating.

6. The medical swab of claim 4, wherein the treatment comprises an adhesive that acts as a hydrophobic coating.

7. The medical swab of claim 6, wherein the adhesive is also used to adhere the first portion of the swab tip to an inner wall of the hollow tube shaft.

8. The medical swab of claim 1, further comprising a crimp formed in the hollow tube shaft to form a break-point, wherein the break-point comprises a weak portion of the hollow tube shaft at which the hollow tube shaft can be broken to reduce its length.

9. The medical swab of claim 1, further comprising a plunger rod positioned within a portion of the hollow tube shaft such that the plunger rod extends outward from the proximal end of the hollow tube shaft.

10. The medical swab of claim 9, wherein plunger rod contacts the first portion of the swab tip and pushes the first portion of the swab tip out the distal end of the hollow tube shaft in response to movement of the plunger rod toward the distal end of the hollow tube shaft.

11. The medical swab of claim 1, wherein the first portion of the swab tip is folded a plurality of times prior to insertion into the distal end of the hollow tube shaft.

12. The medical swab of claim 11, wherein the first portion of the swab tip is folded along a principle thread direction of a material that forms the swab tip.

13. A method of making a medical swab, the method comprising:
shaping at least a portion of a material to form a first portion of a swab tip;
forming a plurality of strips in an end of the material to form a second portion of the swab tip; and
placing the first portion of the swab tip into a distal end of a hollow tube shaft to form the medical swab.

14. The method of claim 13, wherein shaping at least a portion of the material comprises forming the portion of the material into a cylindrical shape to match an opening at the distal end of the hollow tube shaft.

15. The method of claim 13, further comprising inserting a plunger rod into a proximal end of the hollow tube shaft such that a portion of the plunger rod extends out from the distal end of the hollow tube shaft.

16. The method of claim 13, wherein shaping at least a portion of the material comprises folding the material a plurality of times.

17. The method of claim 16, further comprising identifying a principle thread direction of the material, and wherein the folding is performed along the principle thread direction.

18. The method of claim 13, further comprising applying a treatment to the first portion of the swab tip to reduce absorption by the first portion.

19. The method of claim 18, wherein applying the treatment comprises melting at least a portion of the first portion of the swab tip.

20. The method of claim 18, wherein applying the treatment comprises applying a hydrophobic coating to the first portion of the swab tip.
